(19) United States
(12) Patent Application Publication
(10) Pub. No.: US 2014/0200624 A1
(43) Pub. Date: Jul. 17, 2014

(54) SYSTEM AND METHOD OF TREATING INTRACTABLE EPILEPSY BY STIMULATION

(71) Applicant: Harinder Jaseja, Gwalior (IN)

(72) Inventor: Harinder Jaseja, Gwalior (IN)

(73) Assignee: Harinder Jaseja, Gwalior (IN)

(21) Appl. No.: 14/108,379

(22) Filed: Dec. 17, 2013

Related U.S. Application Data
(60) Provisional application No. 61/752,987, filed on Jan. 16, 2013.

(51) Int. Cl. A61N 1/36

(52) U.S. Cl. CPC A61N 1/36064; A61N 1/36139; A61N 1/36121; A61N 1/36078
USPC 607/45; 607/116

(57) ABSTRACT

System and method for treating intractable epilepsy are provided. The method includes implanting electrodes in pedunculopontine nucleus and delivering electrical pulses to the pedunculopontine nucleus, thereby inducing the stimulation of cholinergic neurons leading to the release of acetylcholine, thereby enhancing the genesis of rapid eye movement sleep, which reduces the occurrence of epileptic attacks and also chronically suppresses the epileptogenic process.

### CROSS REFERENCE TO RELATED APPLICATIONS

This application claims the benefit of U.S. provisional patent application No. 61/752,987 filed on Jan. 16, 2013 the entire disclosure of which is hereby incorporated by reference in its entirety as if set forth verbatim herein and relied upon for all purposes.

### FIELD

In general, subject matter relates to the field of treatment of intractable epilepsy. More particularly, but not exclusively, to treatment of epilepsy using stimulation.

### DISCUSSION OF RELATED FIELD

Epilepsy is a chronic neurological condition that is characterized by recurrent seizures. Anything that disturbs the normal pattern of activity in the brain or imbalance of neurotransmitters in the brain can cause epilepsy.

The primary indication of epilepsy is synchronized electrical activity between large numbers of brain neurons. During a seizure, a group of neurons in the brain demonstrate a highly synchronized firing pattern.

Researchers have found that, epilepsy may occur because of two reasons, high level of excitatory neurotransmitters or abnormally low level of inhibitory neurotransmitters.

Over a period of time, several drugs have been proposed for treating epilepsy. Some of these drugs include, carbamazepine, ethosuximide, felbamate, tiagabine, levetiracetam, lamotrigine, pregabalin, gabapentin, phenytoin, Topamax and oxcarbazepine, among others. However, while these drugs have a positive impact towards treating epilepsy, it has been observed that several patients are not completely controlled by usage of such drugs and fall in the category of drug-resistant or intractable epilepsy. Hence, supplementary techniques to treat intractable epilepsy have been explored.

Some of the supplementary techniques include, but are not limited to, ketogenic diet, Vagal Nerve Stimulation (VNS) and anterior thalamic stimulation, which is a form of Deep Brain Stimulation (DBS).

Ketogenic diet is primarily used to treat intractable epilepsy in children. This diet includes consumption of high-fat, adequate-protein and low-carbohydrate content. However, it has been observed that, ketogenic diet may not work well for all children. Further, this diet is also known to have side effects, such as, constipation, kidney stones, high cholesterol and decreased growth rate in children, among other side effects. Furthermore, this diet is rarely suggested for adults with epilepsy. Lastly, the ketosis-state is very difficult to attain and very easily reversible.

Further, as previously mentioned, VNS is another supplementary method to treat intractable epilepsy. Although this method has been widely adopted to treat intractable epilepsy, there are several disadvantages associated with the technique. Some of the disadvantages include infection from implant surgery, voice changes or hoarseness, cough or sore throat, neck pain, discomfort or tingling in the area where the device is being implanted, breathing problems especially while carrying out physically strenuous tasks, difficulty in swallowing and trouble in remembering information learned shortly after the procedure.

Similarly, thalamic DBS (TDBS) is another supplementary stimulation technique used to treat intractable epilepsy. However, TDBS carries risks associated with any type of brain surgery. For example, the procedure may lead to bleeding in the brain or stroke, infection, disorientation or confusion, mood swings, movement disorders, dizziness and sleeping trouble, among other undesirable effects. Furthermore, TDBS modulates the neural functioning in the brain and its long term and delayed effects are yet to be determined.

The aforementioned conventional methods are still experimental and other side effects, which may occur, are yet to be determined. Further, the therapeutic mechanisms behind these techniques are not clearly known and remain to be elucidated. Furthermore, the aforementioned stimulation techniques have been observed to induce alterations in brain environment.

In light of the foregoing discussion, there is a need for an improved technique to address the aforementioned problems, thereby treating intractable epilepsy.

### OBJECTIVE OF THE INVENTION

An objective of the present invention is to treat intractable epilepsy by electrically stimulating a target site.

Another objective of the present invention is to create a natural antiepileptic environment within the brain to suppress the generation of seizure activity.

Yet another objective of the present invention is to enhance rapid eye movement (REM) sleep during natural sleep by periodic interruption of non rapid eye movement (NREM sleep) by REM sleep.

### SUMMARY

In an embodiment, a system is provided for treating intractable epilepsy. The system includes at least one electrode implanted in pedunculopontine nucleus. Further, a pulse generator is provided to generate electrical pulses, which are delivered to the pedunculopontine nucleus, thereby inducing the stimulation of cholinergic neurons leading to the release of acetylcholine, thereby enhancing the genesis of rapid eye movement sleep.

In another embodiment, a method is provided for treating intractable epilepsy. The method includes implanting at least one electrode in pedunculopontine nucleus and delivering electrical pulses to the pedunculopontine nucleus, thereby inducing the stimulation of cholinergic neurons with release of acetylcholine, thereby enhancing the genesis of rapid eye movement sleep.

### BRIEF DESCRIPTION OF DRAWINGS

Embodiments are illustrated by way of example and not limitation in the Figures of the accompanying drawings, in which like references indicate similar elements and in which:

FIG. 1 illustrates a system 100 for stimulating electric signals to a target site, in accordance with an embodiment.

FIG. 2 illustrates the components of the pulse generator 102 enabling it to generate stimulation in accordance with an embodiment.

FIG. 3 is an exemplary flow chart of configuring the system 100 with the patient's body, in accordance with an embodiment.

### DETAILED DESCRIPTION OF PREFERRED EMBODIMENTS

I. OVERVIEW

II. EXEMPLARY SYSTEM

III. EXEMPLARY MECHANISM OF ACTION

IV. EXEMPLARY METHOD

### I. Overview

In general, subject matter relates to treatment of intractable epilepsy, more particularly, but not exclusively, to treatment of epilepsy using stimulation. In an embodiment, epilepsy is controlled by enhancing the genesis of Rapid Eye Movement (REM) sleep. The REM sleep is induced by stimulation of cholinergic neurons with release of acetylcholine present in a target site by electrically stimulating the target site. Further, the electrical stimulation administered to the target site is controlled according to the patient's need(s).

The following detailed description includes references to the accompanying drawing, which form part of the detailed description. The drawing shows illustration in accordance with example embodiments. These example embodiments are described in enough detail to enable those skilled in the art to practice the present subject matter. However, it will be apparent to one of ordinary skill in the art that the present invention may be practiced without these specific details. In other instances, well-known methods, procedures and components have not been described in detail so as not to unnecessarily obscure aspects of the embodiments. The embodiments can be combined, other embodiments can be utilized or structural and logical changes can be made without departing from the scope of the invention. The following detailed description is, therefore, not to be taken as a limiting sense.

In this document, the terms “a” or “an” are used, as is common in patent documents, to include one or more than one. In this document, the term “or” is used to refer to a nonexclusive “or,” such that “A or B” includes “A but not B,” “B but not A,” and “A and B,” unless otherwise indicated.

### II. First Exemplary System

Epilepsy is considered to occur due to neurological disorder i.e. when the brain activity is in an abnormally synchronized state. It is generally seen that, occurrence of seizures is less in day time as compared to night. The reason being, during day time, patients are alert because of their involvement in different activities, which leads to desynchronized brain activity.

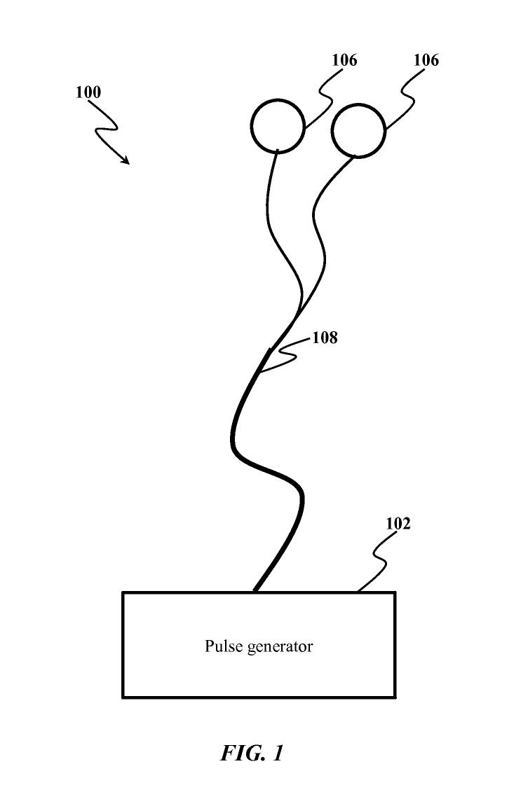

FIG. 1 illustrates a system for stimulating a target site using electric signals, in accordance with an embodiment. The system 100 includes a pulse generator 102, electrodes 106 and a connecting wire 108. The system 100 is used to treat intractable epilepsy.

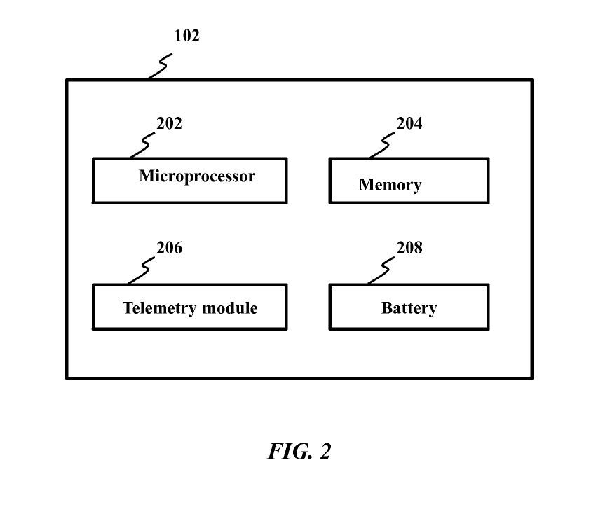

The pulse generator 102 is configured to generate electrical pulses, which are delivered to the electrodes 106. FIG. 2 illustrates the components of the pulse generator 102 enabling it to generate stimulation. In an embodiment, the pulse generator 102 comprises of a microprocessor 202, memory 204, telemetry module 206 and a battery 208.

In an embodiment, the memory 204 stores program instructions for execution by the microprocessor 202. The memory 204 stores the pulse width value, signal amplitude value, pulse frequency value, signal ON time and Signal OFF time, which can be programmed, for example by a medical practitioner.

The microprocessor 202 controls the pulse generator 102 to deliver electrical signals, e.g., as stimulation pulses or continuous waveforms, with current amplitudes, pulse widths and frequency.

The battery 208 serves as a power source to the pulse generator 102. The battery 208 may be rechargeable or non rechargeable battery 208.

Further in an embodiment, the pulse generator 102 includes a telemetry module 206, which further comprises of a wireless transmitter and an antenna for transmission of signals to an external device. The transmitter and antenna is configured to send and receive data and programming and control instructions from an external communication device. The external device for example can be a computer or communication device. The communication device sends the data and receives it wirelessly via bluetooth, wireless transmission protocol.

In an embodiment, the parameters of the electrical pulse to be generated by the pulse generator 102 are pre-configurable. Further, the duration of time during which the electrical pulses have to be generated is configurable. Furthermore, the time intervals during which the electrical pulses have to be generated are configurable.

The electrodes 106 are positioned at the target site to deliver electrical stimulation for limiting the occurrence of intractable epileptic attacks. The electrodes 106 are implanted in the pedunculopontine nucleus (PPN). Note that a single electrode 106 can be used.

In an embodiment, the electrodes 106 are implanted in the target site by conventional surgical method.

In an embodiment, the electrodes 106 receive electric pulses from the pulse generator 102. The electrodes 106 are connected to the pulse generator 102 using the connecting wire 108.

In an embodiment, the electrodes 106 on receiving the electric pulses from the pulse generator 102 stimulate the target site.

In an embodiment, upon placement of multi contact electrode 106 in the target site, the target site is electrically stimulated, thereby resulting in release of acetylcholine neurons. It shall be noted that, the stimulation is delivered through various combination of contacts of the multi contact electrode 106. For example, a multi contact electrode 106 may have multiple region of contact, any one of the region can be selected and thereby be stimulated using a pulse generator 102.

In an embodiment, the electrical stimulation pulses are delivered using at least two contacts of the multi contact electrode 106.

In another embodiment, the electrical stimulation is delivered through at least one contact of the multi contact electrode 106.

In an embodiment, the system 100 further includes medium to initiate stimulation based on a patient's desire. Such medium, for example, can be a magnet, which can be brought to the proximity of the pulse generator 102 to initiate stimulation.

In an embodiment, the targeted treatment site is pedunulopontine nucleus (PPN), which is the major brain stem motor area and controls muscle tone, rigidity, posture, balance, locomotion, and REM sleep. The PPN consists of two subdivisions pars compacta and pars dissipata. The pars compacta consist of cholinergic neurons (acetylcholine), which are clustered along the dorsolateral border of the superior cerebellar peduncle (SP) at trochlear nucleus levels.

The cholinergic neurons in the pars compacta division of PPN are not only involved in the maintenance of arousal state but also in generation of REM sleep. It shall be noted that, the pars compacta division of PPN constitutes 90% of the cholinergic neurons.

Synchronization and de-synchronization of brain activity result in increased occurrence and resistance of epileptic seizures, respectively. It shall be noted that, in human sleep wake cycle, 75-89% of total night sleep time constitutes Non Rapid Eye Movement (NREM) sleep. The brain activity is in synchronised state during this stage of sleep. Therefore, the probability of seizure occurrence is more during night time. However, in the day time the brain activity tends to be in de-synchronized state promoting resistance to occurrence of seizures.

Further in an embodiment, the stimulation of PPN is carried out during night time. The stimulation of PPN by the pulse generator 102 generates electric impulses. The generation of these electric pulses induces the release of cholinergic neurons, thereby enhancing the genesis of REM sleep.

In an embodiment, the sleep stage of a patient is determined to enhance the REM sleep occurrence during the night time. In an embodiment, the sleep stage may be determined by performing Polysomnography (PSG). The method of detecting sleep stage by PSG is well known in the art and hence not needed to be described. Examples of biosignals indicative of activity within a brain of a patient include, but not limited to, EEG signal or an ECoG signal. Information corresponding to sleep stage of a patient is received by the system 100, which stimulates PPN with electric pulses when the patient is in NREM sleep. It shall be noted that stimulation is not provided each time NREM sleep is detected. Stimulation is provided as per pre-configured dosage to increase genesis of REM sleep, based on patient's condition and requirement.

In an embodiment, in order to reduce the occurrence of intractable epileptic attacks, the NREM sleep cycle is interrupted and disrupted periodically, thereby converting the NREM sleep into REM sleep. However, it shall be noted that, NREM sleep is not completely converted to REM sleep. It is only periodic interruption of NREM sleep phase by REM sleep, thereby enhancing the occurrence of REM sleep during the night time, thereby weakening the influences and forces that favor or promote epileptogenesis.

In an embodiment, the system 100 will be operational (i.e. at switch-on mode) during the night sleep time to enhance the protective influence of REM sleep (PPN stimulation enhances REM sleep) thus offering and strengthening the natural innate protection against epileptogenesis.

In another embodiment, the stimulation of PPN may be carried out during daytime in accordance to the need of the patient. It shall be noted that, stimulation of low frequency (10 Hz to 25 Hz) is delivered to the target site in daytime.

### III. Exemplary Mechanism of Action

Acetylcholine plays a vital role in inducing REM sleep. It has been observed that, the release of acetylcholine is highest during waking and REM sleep. This is because the initiation of REM sleep begins in the acetylcholine neurons located in the pons specifically in Peribrachial area.

Further, REM sleep is regarded as the most potent antiepileptic state during human wake-sleep cycle. Occurrence of epileptic seizures is minimum during REM sleep. In order to weaken the influences and forces that promote epileptogenesis, the enhancement of REM sleep is essential. It shall be noted that, cholinergic neurons i.e. acetylcholine in the PPN are involved in generation of REM sleep.

The above mentioned technique creates a natural environment (REM sleep state) in the brain, which is potentially the most potent method to treat intractable epilepsy.

### IV. Exemplary Method

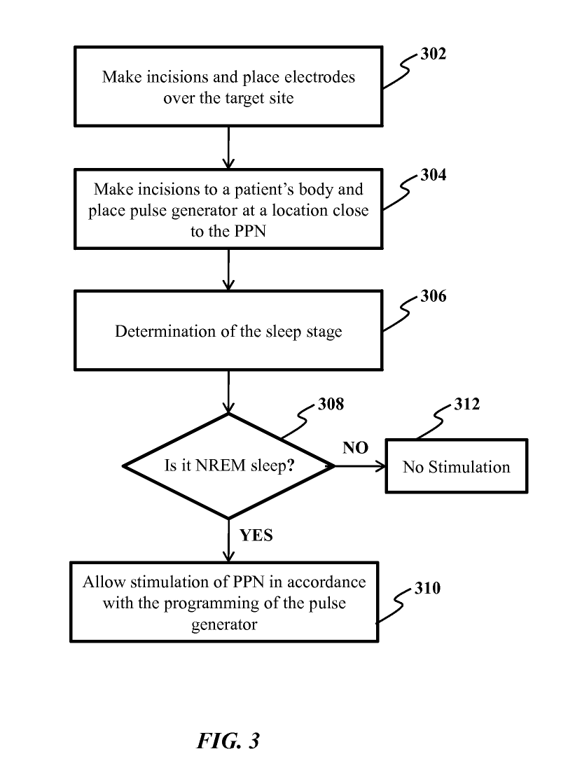

FIG. 3 is an exemplary flow chart of configuring the system 100 with the patient's body, in accordance with an embodiment. At step 302, incisions are made to the patient's body, such that electrodes 106 can be implanted over the target site. At step 304, incisions to a patient's body are made, and pulse generator 102 is placed close to the target site (PPN). The pulse generator 102, for example, can be implanted under the collar bone or on the upper left side of the chest. At step 306, the sleep stage of a patient is determined. If the sleep stage is determined to be NREM sleep (at step 308), then electrical stimulation pulses are delivered to the electrode 106 (at step 310). Further, at step 312, no electrical stimulation is delivered, if the sleep stage is determined to be REM sleep (at step 308).

The processes described above are described as sequence of steps, this was done solely for the sake of illustration. Accordingly, it is contemplated that some steps may be added, some steps may be omitted, the order of the steps may be re-arranged, or some steps may be performed simultaneously.

Although embodiments have been described with reference to specific example embodiments, it will be evident that various modifications and changes may be made to these embodiments without departing from the broader spirit and scope of the system and method described herein. Accordingly, the specification and drawings are to be regarded in an illustrative rather than a restrictive sense.

Many alterations and modifications of the present invention will no doubt become apparent to a person of ordinary skill in the art after having read the foregoing description. It is to be understood that the phraseology or terminology employed herein is for the purpose of description and not of limitation. It is to be understood that the description above contains many specifications; these should not be construed as limiting the scope of the invention but as merely providing illustrations of some of the personally preferred embodiments of this invention. Thus the scope of the invention should be determined by the appended claims and their legal equivalents rather than by the examples given.

What is claimed is:

1. A system for treating intractable epilepsy, the system comprises:
at least one electrode implanted in pedunculopontine nucleus; and
a pulse generator to generate electrical pulses, which are delivered to the pedunculopontine nucleus, thereby inducing the stimulation of cholinergic neurons leading to the release of acetylcholine, thereby enhancing the genesis of rapid eye movement sleep.

2. The system according to claim 1, wherein parameters of the electrical pulses generated by the pulse generator are pre-configurable.

3. The system according to claim 1, wherein the system is configured to receive information corresponding to sleep stage of a patient and stimulate pedunculopontine nucleus with electric pulses when the patient is in non-rapid eye movement sleep, to increase genesis of REM sleep, wherein the stimulation is provided in pre-configured dosage based on patient's condition.

4. A method for treating intractable epilepsy, the method comprises:
implanting at least one electrode in pedunculopontine nucleus; and
delivering electrical pulses to the pedunculopontine nucleus, thereby inducing the release of cholinergic neurons, thereby enhancing the genesis of rapid eye movement sleep.

5. The method according to claim 4, wherein the electrical pulses are generated by a pulse generator.

6. The method according to claim 5, wherein the pulse generator is implanted under the collar bone or on the upper left side of the chest.

7. The method according to claim 4, wherein the delivery of electrical pulses is pre-configurable.

8. The method according to claim 4, further comprising receiving information corresponding to sleep stage of a patient and stimulating pedunculopontine nucleus with electric pulses when the patient is in non-rapid eye movement sleep, to increase genesis of REM sleep, wherein the stimulation is provided in pre-configured dosage based on patient's condition.
